SEQUENCE LISTING (4 sequences)

SEQ ID NO: 1 - protein, 164 aa, Artificial Sequence
  Truncated Canine sequence
CHLPDTHGLRNWRVLTLLGQMRRLSAGSCDHYTNDFAFPKELFDGQRLQEAQALSVVHVMTQKVFHLFCPDTSSAPWNMTLLEELCSGLSEQLDDLEACPLQEAGLAETPLMHEDSTLRTYFQRISLYLQDRNHSPCAWEMVRAEIGRSFFSSTILQERIRRRK

SEQ ID NO: 2 - protein, 164 aa, Artificial Sequence
  hybrid canine sequence
CDLPDTHSLRNWRVLTLLGQMRRLSASSCDHYTTDFAFPKELFDGQRLQEAQALSVVHVMTQKVFHLFCPDTSSAPWNMTLLEELCSGLSEQLDDLDACPLQEAGLAETPLMHEDSTLRTYFQRISLYLQDRNHSPCAWEMVRAEIGRSFFSLTILRERIRRRK

SEQ ID NO: 3 - protein, 187 aa, Canis lupus
MALPCSFSVALVLLSCHSLCCLACHLPDTHSLRNWRVLTLLGQMRRLSASSCDHYTTDFAFPKELFDGQRLQEAQALSVVHVMTQKVFHLFCTNTSSAPWNMTLLEELCSGLSEQLDDLDACPLQEAGLAETPLMHEDSTLRTYFQRISLYLQDRNHSPCAWEMVRAEIGRSFFSLTILQERVRRRK

SEQ ID NO: 4 - protein, 187 aa, Canis lupus
MALPCSFSVALVLLSCHSLCCLACHLPDTHGLRNWRVLTLLGQMRRLSAGSCDHYTNDFAFPKELFDGQRLQEAQALSVVHVMTQKVFHLFCPDTSSAPWNMTLLEELCSGLSEQLDDLEACPLQEAGLAETPLMHEDSTLRTYFQRISLYLQDRNHSPCAWEMVRAEIGRSFFSSTILQERIRRRK

(19) United States
(12) Patent Application Publication
(10) Pub. No.: US 2024/0010698 A1
(43) Pub. Date: Jan. 11, 2024

(54) COMPOSITIONS AND METHODS RELATING TO THE TREATMENT OF DISEASES

(71) Applicant: ILC THERAPEUTICS LIMITED, Lanarkshire (GB)

(72) Inventors: William STIMSON, Lanarkshire (GB); Christopher MCKENZIE, Lanarkshire (GB)

(73) Assignee: ILC THERAPEUTICS LIMITED, Lanarkshire (GB)

(21) Appl. No.: 18/018,642

(22) PCT Filed: Jul. 30, 2021
(86) PCT No.: PCT/GB2021/051978

§ 371 Date: Jan. 30, 2023

(30) Foreign Application Priority Data
Jul. 31, 2020 (GB) 2011945.9

(51) Int. Cl. C07K 14/56; C12N 15/70; A61P 17/04

(52) U.S. Cl. CPC C07K 14/56; C12N 15/70; A61P 17/04

(57) ABSTRACT

The present invention relates to compositions and methods for preventing or treating canine pruritic disease, optionally wherein the canine pruritic disease is canine atopic dermatitis (AD) in general, and a synthetic canine interferon (IFN)-alpha (α) for use in said treatment or prophylaxis in particular, as well as a composition and method comprising the same and a method of production of the synthetic canine interferon-alpha.

### FIELD OF THE INVENTION

The present invention relates to compositions and methods for preventing or treating canine pruritic diseases, in particular canine atopic dermatitis (AD). Canine interferon and in particular a synthetic canine interferon (IFN)-alpha (α) for use in said treatment or prophylaxis is provided, as well as a composition and method comprising the same and a method of production of the synthetic canine interferon-alpha.

### BACKGROUND TO THE INVENTION

Allergic pruritic diseases are increasingly common in dogs. These are characterised by dermatosis with intense pruritus and inflammation. Atopic dermatitis (AD) is the primary variety of allergic skin conditions.

Canine atopic dermatitis (AD) is a common condition in dogs resulting in dry skin, erythema and self-induced excoriations, commonly at the scalp, face, neck, and flexural surfaces of the extremities. Canine AD occurs due to excessive immune responses of the CD4 Th2 “phenotype”. These are usually due to genetic predisposition, but environmental factors may be a determining factor. The activation of this immune pathway leads to IgE production and allergy-like reactions in the skin and requires long-term treatment.

Standard-of-care therapies include the use of anti-histamines, corticoids and, more recently, a small-molecule inhibitor, oclacitinib—see T. Olivry et al. “Treatment of canine atopic dermatitis: 2015 updated guidelines from the International Committee on Allergic Diseases of Animals (ICADA)”, BMC Vet. Res. 11 (2015) 210 which is herein incorporated by reference.

In Japan, canine interferon-gamma has been used in the treatment of AD. The goal of the therapy is to revert the Th1/Th2 immune unbalance that leads to excessive IgE responses—see K. Yasukawa et al. “Low-dose recombinant canine interferon-γ for treatment of canine atopic dermatitis: An open randomized comparative trial of two doses”, Vet. Dermatol. 21 (2010) 42-49.3,4] which is herein incorporated by reference.

Recombinant human interferon-alpha14 (rhIFNa-14) has also been used in the treatment of canine allergic dermatitis—see WO2019/229480 and “Recombinant human interferon-α14 for the treatment of canine allergic pruritic disease in eight dogs” by Beirio et al., Vet Record Open, Vol. 8, Issue 1, May 2021—both of which are herein incorporated by reference. However, use of human interferon-alpha14 led to inflammation, skin rashes, fever, vomiting and nausea in dogs when injected. Additionally, some dogs were non-responsive to the treatment and had higher titres of anti-recombinant human IFNα14 than responsive dogs.

As discussed in for example “Atopic dermatitis in cats and dogs: a difficult disease for animals and owners” by Gedon and Mueller Clinical Translational Allergy (2018) 8:41 (which is herein incorporated by reference), whilst some similarities exist between human and canine AD there are also differences. Several functional and truncated interferon-alpha pseudogenes are considered to exist in the canine genome; however, the role of each of the expressed interferons in canine is not clearly understood. Several of these canine sequences share sequence identity of about 52 to 55% to human interferon-alpha sequences.

Allergic dermatitis hampers life quality in dogs as pruritus progressively interferes with normal behaviours. Accordingly, there is a need for new effective treatments for the treatment of Canine AD.

### SUMMARY OF THE INVENTION

The present invention provides a canine interferon-alpha for use in the treatment or prophylaxis of allergic pruritic diseases and in particular canine atopic dermatitis, wherein the canine interferon-alpha comprises or consists of an amino acid sequence SEQ ID NO: 1 or a functionally active fragment or variant thereof.

SEQ ID NO: 1
CHLPDTHGLRNWRVLTLLGQMRRLSAGSCDHYTNDFAFPKELFDGQRLQE
AQALSVVHVMTQKVFHLFCPDTSSAPWNMTLLEELCSGLSEQLDDLEACP
LQEAGLAETPLMHEDSTLRTYFQRISLYLQDRNHSPCAWEMVRAEIGRSF
FSSTILQERIRRRK

The inventor has determined that an amino acid sequence SEQ ID NO: 1 or a functionally active fragment or variant thereof produces a functional effect of an improved effect on the inhibition of IL-17A and GM-CSF compared to human interferon-alpha-14 when tested on whole canine blood.

In particular, canine interferon-alpha of the invention was found to outperform human IFNα14 in relation to the effect on the inhibition of IL-17.

It is considered that a functionally active variant of SEQ ID NO: 1 may comprise of substitution mutations of amino acids based on other canine interferon-alpha sequences. Suitably a variant may be provided in which SEQ ID NO: 1 includes substitution mutations based on a consensus of interferon-alpha sequences from dogs, for example at least the interferon-alpha sequence from dogs (IFN alpha 1/2 (Uniprot P81255) (SEQ ID NO:4), and Interferon-alpha 3 from dogs (097945) (SEQ ID NO: 3), both of which are herein incorporated by reference,

(SEQ ID NO: 3)
MALPCSFSVALVLLSCHSLCCLACHLPDTHSLRNWRVLTLLGQMRRLSAS
SCDHYTTDFAFPKELFDGQRLQEAQALSVVHVMTQKVFHLFCTNTSSAPW
NMTLLEELCSGLSEQLDDLDACPLQEAGLAETPLMHEDSTLRTYFQRISL
YLQDRNHSPCAWEMVRAEIGRSFFSlTILQERVRRRK

and/or a consensus sequence based on at least three, four, five, six, seven, eight, nine, ten, 11 or 12 interferon sequences from dog. Suitably a variant may comprise or consist of an amino acid sequence as set out in SEQ ID NO: 2 and/or SEQ ID NO: 3.

For example, suitably, a variant may comprise one or more substitutions at one or more points of the sequence of SEQ ID NO: 1 where variability between IFN alpha sequences are observed. For example suitably a variant may comprise a substitution at one or more positions selected from the group consisting of: H2, G8, G27, N34, E97, S153, and Q157, wherein the position numbering is defined as that position which when aligned based on primary structure corresponds to the same position of the interferon shown herein as SEQ ID NO: 1. As will be appreciated, for example in the alignment of SEQ ID NO: 1 and SEQ ID NO: 3 shown in FIG. 10 B, then numbering of SEQ ID NO: 1 would cause G8 to be indicated at position 31 on the alignment—95.1% sequence identity when using comparison matrix BLOSUM62, GAP OPEN PENALTY: 12 and GAP EXTENSION PENALTY: 4.

Suitably, the variant may comprise or consist of an amino acid sequence having at least 90% identity, at least 95%, at least 97%, at least 99% sequence identity to SEQ ID NO: 1 or a functionally active fragment of SEQ ID NO: 1.

Suitably a variant of SEQ ID NO: 1 may be provided to enhance the expression of the amino acid sequence in an E. coli production system. Suitably a variant of SEQ ID NO: 1 may be provided to enhance the stability of the amino acid sequence in a preparation or formulation to be administered to the canine. Suitably, a variant of SEQ ID NO: 1 may be provided to enhance the solubility of the amino acid in a preparation or formulation to be administered to the canine.

Suitably the amino acid sequence with a sequence as set forth in SEQ ID NO: 1 or a variant of fragment thereof is provided to a canine as a sub lingual preparation or formulation. For examples, suitably the amino acid sequence is provided in a saline solution for administration to a canine by a sublingual route. Suitably, treatment of the canine may be at regular intervals, for example every few weeks (3 to 4 weeks between administrations).

Suitably, the variant may comprise or consist of an amino acid sequence shown as SEQ ID NO: 2 or a functionally active fragment of SEQ ID NO: 2.

SEQ ID No. 2:
CDLPDTHSLRNWRVLTLLGQMRRLSASSCDHYTTDFAFPKELFDGQRLQE
AQALSVVHVMTQKVFHLFCPDTSSAPWNMTLLEELCSGLSEQLDDLDACP
LQEAGLAETPLMHEDSTLRTYFQRISLYLQDRNHSPCAWEMVRAEIGRSF
FSLTILRERIRRRK

Sequence identity of SEQ ID NO: 1 and 2 as shown in FIG. 10 A—95.7%, when using comparison matrix BLOSUM62, GAP OPEN PENALTY: 12 and GAP EXTENSION PENALTY: 4.

Suitably, the canine interferon-alpha may be expressed in E. coli. It will be appreciated by those skilled in the art that the success of expressing eukaryotic proteins in prokaryotic cells (as is the case here) is highly unpredictable. Mis-folding, degradation and packaging into inclusion bodies is common, due to the differences between the different cell types in how proteins are folded and modified and the differences between transport proteins, expression tags and cellular machinery. Alternatively, even if a eukaryotic protein is correctly folded in a prokaryotic cell, the prokaryotic cell may have left markers (e.g. post-translational modifications) on the surface of the protein. These may modify or ameliorate its intended biological activity. Common solutions are known in the art, such as sequence modification, purification tags, refolding the protein (which is expensive, time consuming and stochastically successful), etc. The inventors have therefore surprisingly found that expression of the canine interferon-alphas of the present invention in E. coli results in canine interferon-alphas with the desired activity.

Suitably, the canine interferon-alpha or a variant or fragment as discussed herein may be formulated for oral, sublingual or subcutaneous administration.

In another aspect, the present invention relates to a pharmaceutical composition for use in the treatment of canine allergic pruritic disease, in particular canine atopic dermatitis, wherein the composition comprises a canine interferon-alpha of the present invention and a pharmaceutically acceptable carrier, diluent or excipient.

In a further aspect, the present invention relates to a method of or for the treatment and/or prophylaxis of canine allergic pruritic disease, in particular canine atopic dermatitis, said method comprising the step of:
(i) administering to a canine in need thereof a therapeutically effective amount of a canine interferon-alpha comprising or consisting of an amino acid sequence SEQ ID NO:1 or a functionally active fragment or variant thereof.

Suitably, the variant may comprise or consist of an amino acid sequence having at least 90% identity, at least 95%, at least 97%, at least 99% sequence identity to SEQ ID NO: 1 or a functionally active fragment of SEQ ID NO: 1.

Suitably, the variant may have substitutions at one or more positions selected from the group consisting of: H2, G8, G27, N34, E97, S153, and Q157, wherein the position numbering is defined as that position which when aligned based on primary structure corresponds to the same position as that of SEQ ID NO: 1.—see FIG. 10A.

SEQ ID NO: 1
CHLPDTHGLRNWRVLTLLGQMRRLSAGSCDHYTNDFAFPKELFDGQRLQE
AQALSVVHVMTQKVFHLFCPDTSSAPWNMTLLEELCSGLSEQLDDLEACP
LOEAGLAETPLMHEDSTLRTYFQRISLYLQDRNHSPCAWEMVRAEIGRSF
FSSTILQERIRRRK

Suitably, the variant may comprise or consist of an amino acid sequence shown as SEQ ID NO: 2 or a functionally active fragment of SEQ ID NO: 2 (amino acid substitutions from SEQ ID NO: 1 shown in bold).

SEQ ID No. 2:
CDLPDTHSLRNWRVLTLLGQMRRLSASSCDHYTTDFAFPKELFDGQRLQE
AQALSVVHVMTQKVFHLFCPDTSSAPWNMTLLEELCSGLSEQLDDLDACP
LQEAGLAETPLMHEDSTLRTYFQRISLYLQDRNHSPCAWEMVRAEIGRSF
FSLTILRERIRRRK

Suitably, the canine interferon-alpha may be expressed in E. coli.

Suitably, the canine interferon-alpha may be administered orally or by subcutaneous administration.

In a further aspect, the present invention provides a method for the production of a canine interferon-alpha comprising the steps of:
(i) providing an Escherichia coli cell;
(ii) transforming the Escherichia coli cell with a heterologous nucleotide sequence encoding a canine interferon-alpha under the control of a promoter sequence wherein the canine interferon-alpha has at least 90% identity to SEQ ID NO:1 or a functionally active fragment of SEQ ID NO: 1; and
(iii) expressing the canine interferon-alpha in the cell.

In another aspect, there is provided a canine interferon-alpha for use in the treatment or prophylaxis of canine allergic pruritic disease, in particular canine atopic dermatitis, wherein the canine interferon-alpha is produced by the method for the production of a canine interferon-alpha of the invention.

In a further aspect, the present invention provides a method of or for the treatment and/or prophylaxis of canine allergic pruritic disease, in particular canine atopic dermatitis, said method comprising the step of:
(i) administering to a canine in need thereof a therapeutically effective amount of a canine interferon-alpha produced by the method for the production of a canine interferon-alpha of the invention.

### DETAILED DESCRIPTION OF THE INVENTION

Human interferon-alpha 14 has been used to treat atopic dermatitis in dogs. However, such use was determined to cause inflammation when injected in some dogs. The inventor of the present invention has surprisingly discovered that a fragment of a representative canine interferon-alpha 1/2 can be administered to dogs without adverse effects and surprisingly that the fragment shown as SEQ ID No. 1 is particularly active against canine IL-17a and GM-CSF. SEQ ID No. 1 is an E. coli recombinant of Canine alpha 1/2 having a 23 amino acid truncation of the representative sequence shown in the Uniprot database having accession number P81255, which is herein incorporated by reference.

>P81255 (SEQ ID NO: 4 including bold portion
in addition to SEQ ID NO: 1
MALPCSFSVALVLLSCHSLCCLACHLPDTHGLRNWRVLTLLGQMRRLSAG
SCDHYTNDFAFPKELFDGQRLQEAQALSVVHVMTQKVFHLFCPDTSSAPW
NMTLLEELCSGLSEQLDDLEACPLQEAGLAETPLMHEDSTLRTYFQRISL
YLQDRNHSPCAWEMVRAEIGRSFFSSTILQERIRRRK

In particular, SEQ ID No. 1 omits the first 23 amino acids of the representative sequence P81255, which is considered to be a signal sequence.

Suitably, it may be considered that a similar initial sequence may be provided by SEQ ID NO: 3 and suitably fragments of SEQ ID NO: 3 which do not contain this corresponding sequence may be provided.

The invention has surprisingly also found that variants up having 90% or more identity to SEQ ID No. 1, and 4 can also be generated having similar inhibitory effects on all cytokines tested.

Pruritus in canines is often derived from immune-mediated processes—allergic pruritus; however, itch may arise from neurological or psychological conditions.

Suitably the provision of the interferons of the present invention would not benefit canines with itch from neurological or psychological conditions.

Canine Interferon-Alpha for Use in the Treatment or Prophylaxis of Canine Atopic Dermatitis

Accordingly, in a first aspect the present invention provides a canine interferon-alpha for use in the treatment or prophylaxis of canine atopic dermatitis, wherein the canine interferon-alpha comprises or consists of an amino acid sequence SEQ ID NO: 1 or a functionally active fragment or variant thereof.

Suitably, the variant may have substitutions at one or more positions selected from the group consisting of: H2, G8, G27, N34, E97, S153, and Q157, wherein the position numbering is defined as that position which when aligned based on primary structure corresponds to the same position of the amino acid sequence shown herein as SEQ ID NO: 1. Sequence alignment as a tool for comparing two or more amino acid or DNA sequences is well known in the art, particularly with such tools as Clustal Omega®.

Suitably, the variant may have at least 2 or at least three or at least 4 or at least 5 or at least six substitutions from the group consisting of: H2, G8, G27, N34, E97, S153, and Q157, wherein the position numbering is defined as that position which when aligned based on primary structure corresponds to the same position of the amino acid sequence shown herein as SEQ ID NO: 1. Suitably, the variant may substitutions at all seven positions.

Suitably, if the variant has substitutions at positions other than H2, G8, G27, N34, E97, S153, and Q157, wherein the position numbering is defined as that position which when aligned based on primary structure corresponds to the same position of the amino acid sequence shown herein as SEQ ID NO: 1, such substitutions are conservative substitutions and the variant retains is a functionally active variant of SEQ ID No. 1.

Suitably, the at least one or more substitutions at positions H2, G8, G27, N34, E97, S153 and Q157 may be selected from:
i) H2D or an equivalent conservative substitution for Aspartic Acid or Histidine,
ii) G8S or an equivalent conservative substitution for Glycine or Serine,
iii) G27S or an equivalent conservative substitution for Glycine or Serine,
iv) N34T or an equivalent conservative substitution for Asparagine or Tyrosine,
v) E97D or an equivalent conservative substitution for glutamic acid of aspartic acid,
vi) S153L or an equivalent conservative substitution for serine of lysine and
vii) Q157R or an equivalent conservative substitution for glutamine and arginine,
wherein the position numbering is defined as that position which when aligned based on primary structure corresponds to the same position of the amino acid sequence shown herein as SEQ ID NO: 1.

Suitably, the variant may have at least one or more of the following substitutions a H2D, G8S, G27S, N34T, E97D, S153L and Q157R, wherein the position numbering is defined as that position which when aligned based on primary structure corresponds to the same position of the amino acid sequence shown herein as SEQ ID NO: 1. The inventors have demonstrated that all of these substitutions can be made whilst retaining the functional activity of SEQ ID No. 1. As such, a person of ordinary skill in the art would recognise that any combination of such substitutions could be made whilst retaining the functional activity of SEQ ID No. 1.

Suitably, the variant may have the amino acid sequence of SEQ ID No. 2. FIGS. 5 to 9 demonstrate that this variant is a functionally active variant of SEQ ID No. 1.

Suitably, the functionally active fragment or variant of the canine interferon-alpha comprising or consisting of an amino acid sequence SEQ ID NO: 1 has the similar and improved activity IL-17A and/or GM-CSF. The inventor has surprising found that a canine interferon-alpha having the amino acid sequence of SEQ ID No.1 or a functionally active variant, such as the synthetic canine interferon-alpha having the amino acid sequence of SEQ ID No. 2 has an improved effect on the inhibition of IL-17A and GM-CSF compared to human interferon-alpha-14 when tested on whole beagle blood—see FIGS. 2 and 3 for the comparison between human interferon-alpha-14 and SEQ ID No. 1 and see FIGS. 5 and 6 showing that SEQ ID No. 2 has similar activity to SEQ ID No. 1 on IL-17A and GM-CSF.

Atopic dermatitis is a Th2-associated disease and involves increases in IL-17A. There are also significant increases in chemokines such as GM-CSF, which are involved in granulocyte proliferation and attraction. Advantageously, the inventor has shown that utilising a Canine interferon-alpha such as that shown in SEQ ID No.1 or a functionally active variant such as that shown in SEQ ID No. 2 reduces the increase in IL-17A more effectively than an equivalent dose of human interferon-alpha-14. For example, the IC₅₀ dose for human interferon-alpha-14 in beagle whole blood is 1×10⁴IU where the IC₅₀ using the canine interferon-alpha of SEQ ID NO. 1 is just 1×10² IU. Likewise, the Canine interferon-alpha is more effective at reducing the increase of GM-CSF associated with canine dermatitis particularly at higher doses.

Suitably, the canine interferon-alpha may be expressed in E. coli.

Suitably, the canine interferon-alpha may be formulated for appropriate delivery. For example, via oral or subcutaneous administration. Suitably delivery may be parenterally or topically or any other suitable means in the art.

Suitably, the canine interferon-alpha may be formulated for administration at a dose of 50 IU/ml, 1×10² IU/ml, 1×10³ IU/ml, 1×10⁴ IU/ml, 1×10⁵IU/ml or 1×10⁶ IU/ml. Suitably, the canine interferon-alpha may be formulated for administration at a dose of 0.1 mg to 1 mg, 1 mg to 3 mg, 3 mg to 5 mg or 5 mg to 10 mg. For example, in dogs sublingual use may be 10⁴IU/Kg, for example in 1 ml PBS.

Suitably, the canine interferon-alpha may be topically administered once a day, twice a day, three times a day or four times a day. Typically, for sublingual administration, the dose would be provided once a day.

In another aspect, the present invention relates to a pharmaceutical composition for use in the treatment of canine allergic pruritic disease, suitably canine atopic dermatitis, wherein the composition comprises a canine interferon-alpha of the present invention and a pharmaceutically acceptable carrier, diluent or excipient.

Pharmaceutical Compositions

Pharmaceutical compositions according to the present invention, and for use in accordance with the present invention, may comprise, in addition to an active ingredient, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be, for example, oral, intravenous, intranasal or via oral or nasal inhalation. The formulation may be a liquid, for example, a physiologic salt solution containing non-phosphate buffer at pH 6.8-7.6, or a lyophilised or freeze-dried powder.

Method for the Treatment and/or Prophylaxis of Canine Atopic Dermatitis

In a further aspect, the present invention relates to a method for the treatment and/or prophylaxis of canine allergic pruritic disease, suitably canine atopic dermatitis, said method comprising the step of:
(i) administering to a canine in need thereof a therapeutically effective amount of a canine interferon-alpha comprising or consisting of an amino acid sequence SEQ ID NO:1 or a functionally active fragment or variant thereof.

Suitably, the variant may comprise or consist of an amino acid sequence having at least 90% identity to SEQ ID NO: 1 or a functionally active fragment of SEQ ID NO: 1.

Suitably, the variant may be any variant described herein. Variants as described for use in the treatment of allergic pruritic disease and in particular Atopic dermatitis above, may also be utilised in the method of treatment aspects.

Suitably, the canine interferon-alpha may be administered orally or by subcutaneous administration. Suitably the interferon of the present invention may be by sublingual administration to the canine to be treated.

Production of Canine Interferon-Alpha

In a further aspect, the present invention provides a method for the production of a canine interferon-alpha comprising the steps of:
(i) providing an Escherichia coli cell;
(ii) transforming the Escherichia coli cell with a heterologous nucleotide sequence encoding a canine interferon-alpha under the control of a promoter sequence wherein the canine interferon-alpha has at least 90% identity to SEQ ID NO:1 or a functionally active fragment of SEQ ID NO: 1; and
(iii) expressing the canine interferon-alpha in the cell.

Suitably, the canine interferon-alpha may be a functionally active variant of SEQ ID No. 1.

Suitably, the heterologous nucleotide sequence encoding a canine interferon-alpha may encode an amino acid sequence having at least at least 91% or at least 92% or at least 94% or at least 95%, or at least 96% or at least 97% or at least 98% or at least 99% sequence identity with SEQ ID NO: 1.

Suitably, the heterologous nucleotide sequence encoding a canine interferon-alpha may encode a functionally active variant of SEQ ID No. 1, wherein the variant is any variant as described herein. Suitably, the heterologous nucleotide sequence encoding a canine interferon-alpha may encode any variant as described for use in the treatment of canine pruritic disease or canine atopic dermatitis above.

In another aspect, there is provided a canine interferon-alpha for use in the treatment or prophylaxis of canine pruritic disease or canine atopic dermatitis, wherein the canine interferon-alpha is produced by the method for the production of a canine interferon-alpha of the invention.

In a further aspect, the present invention provides a method for the treatment and/or prophylaxis of canine pruritic disease or canine atopic dermatitis, said method comprising the step of:
(i) administering to a canine in need thereof a therapeutically effective amount of a canine interferon-alpha produced by the method for the production of a canine interferon-alpha of the invention.

### Definitions

Fragment

A fragment can comprise at least 100, least 115, at least 120 contiguous amino acids from SEQ ID NO: 1, and which is functionally active. Suitably, a fragment may be determined using, for example, C-terminal serial deletion of cDNA. Said deletion constructs may then be cloned into suitable plasmids. The activity of these deletion mutants may then be tested for biological activity as described herein. Fragments may be generated using suitable molecular biology methods as known in the art.

Functionally Active

A fragment or variant of SEQ ID No: 1 that is functionally active is one which has similar or better activity to that of SEQ ID NO: 1 on at least one of more of the following cytokines: IL-17A, GM-CSF, IL-13, IL-8 and IL-31. By similar it is meant that there is no statistically significant differences in response at a defined dose such as at 1×10² IU or 1×10³IU or 1×10⁴IU or 1×10⁵IU or 1×10⁶ IU. Suitably, comparison of activity between the canine interferon-alpha shown as SEQ ID No. 1 and a fragment or variant can be made using beagle leucocytes. Preferably, a variant has similar or improved activity for at least two or at least three or at least four of the cytokines at a particular dose. More preferably, a variant had similar or improved activity for all five cytokines at a particular dose.

Variant

By variant is meant an amino acid sequence which has at least 80% sequence identity to SEQ ID NO: 1. Suitably a variant of the present invention may have at least 90% or at least 91% or at least 92% or at least 94% or at least 95%, or at least 96% or at least 97% or at least 98% or at least 99% sequence identity with SEQ ID NO: 1. A variant encompasses a polypeptide sequence of SEQ ID NO: 1, which includes substitution of amino acids, especially a substitution(s) which is/are known for having a high probability of not leading to any significant modification of the biological activity or configuration, or folding, of the protein. These substitutions, typically known as conserved substitutions, are known in the art. For example the group of arginine, lysine and histidine are known interchangeable basic amino acids. Suitably, in embodiments amino acids of the same charge, size or hydrophobicity may be substituted with each other. Suitably, any substitution may be selected based on analysis of amino acid sequence alignments of interferon-alpha subtypes to provide amino acid substitutions to amino acids which are present in other alpha subtypes at similar or identical positions when the sequences are aligned. Variants may be generated using suitable molecular biology methods as known in the art. Suitably a variant may be based on introducing substitutions from alternative canine interferon-alpha sequences.

Treatment/Therapy

The term “treatment” is used herein to refer to any regimen that can benefit a Canine. The treatment may be in respect of allergic pruritic disease, in particular Atopic Dermatitis and the treatment may be prophylactic (preventative treatment). Treatment may include curative or alleviative effects. Reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition. Accordingly, therapeutic and/or prophylactic treatment includes amelioration of the symptoms of a particular pruritic condition or preventing or otherwise reducing the risk of developing a particular pruritic condition. The term “prophylactic” may be considered as reducing the severity or the onset of a particular condition. “Therapeutic” may also reduce the severity of an existing condition.

Administration

The active ingredients used in the present invention in particular the Canine interferon-alpha shown as SEQ ID No. 1 or a variant or fragment described herein can be administered separately to the same subject, optionally sequentially, or can be co-administered simultaneously as a pharmaceutical or immunogenic composition. The pharmaceutical composition will generally comprise a suitable pharmaceutical excipient, diluent or carrier selected depending on the intended route of administration.

The active ingredients can be administered to a patient in need of treatment via any suitable route. The precise dose will depend upon a number of factors, as is discussed below in more detail.

One suitable route of administration is topically, e.g. applied directly to the skin.

One suitable route is sublingual.

Dose

The composition is preferably administered to an individual in a “therapeutically effective amount” or a “desired amount”, this being sufficient to show benefit to the individual. As defined herein, the term an “effective amount” means an amount necessary to at least partly obtain the desired response, or to delay the onset or inhibit progression or halt altogether the onset or progression of a particular condition being treated. The amount varies depending upon the health and physical condition of the canine being treated, the taxonomic group of the canine being treated, the degree of protection desired, the formulation of the composition, the assessment of the medical situation and other relevant factors. It is expected that the amount will fall in a relatively broad range, which may be determined through routine trials. Prescription of treatment, e.g. decisions on dosage etc., is ultimately within the responsibility and at the discretion of general practitioners, veterinarians, physicians or other medical doctors, and typically takes account of the disorder to be treated, the condition of the individual patient, the site of delivery, the method of administration and other factors known to practitioners. The optimal dose can be determined by vets based on a number of parameters including, for example, age, sex, weight, severity of the condition being treated, the active ingredient being administered and the route of administration. Dosage regimes may be adjusted to provide the optimum therapeutic response and reduce side effects. For example, several divided doses may be administered daily, weekly, monthly or other suitable time intervals or the dose may be proportionally reduced as indicated by the exigencies of the situation.

Unless otherwise defined, all technical and scientific terms used herein have the meaning commonly understood by a person who is skilled in the art in the field of the present invention.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

The present invention will now be exemplified with reference to the following non-limiting figures and examples, which are provided for the purpose of illustration and are not intended to be construed as being limiting on the present invention. Other embodiments of this invention will be apparent to those of ordinary skill in the art in view of this description.

### BRIEF DESCRIPTION OF THE FIGURES

FIG. 1 shows the amino acid sequence of a particular fragment of the representative Canine interferon-alpha 1/2 with Uniprot reference P81255 referred to herein as SEQ ID No. 1

FIG. 2 shows the amino acid sequence of SEQ ID No. 2, which is a synthetic canine interferon-alpha.

FIG. 3 shows a comparison of human interferon-alpha-14 with the canine interferon-alpha shown as SEQ ID No. 1 on the inhibition of IL-17A from whole beagle blood with and without stimulation using PHA. The canine IC50 was considered to be around 100 times better than the human was.

FIG. 4 shows a comparison of human interferon-alpha-14 with the canine interferon-alpha shown as SEQ ID No. 1 on the inhibition of GM-CSF production using a beagle whole blood assay wherein IC50 was considered to be the same.

FIG. 5 shows a comparison of the canine interferon-alpha shown as SEQ ID No. 1 with the synthetic interferon-alpha shown as SEQ ID No. 2 on the inhibition of IL-17A from beagle leucocytes (stimulated with PHA).

FIG. 6 shows a comparison of the canine interferon-alpha shown as SEQ ID No. 1 with the synthetic interferon-alpha shown as SEQ ID No. 2 on the inhibition of GM-CSF by beagle leucocytes (assay not stimulated with any mitogen).

FIG. 7 shows a comparison of the canine interferon-alpha shown as SEQ ID No. 1 with the synthetic interferon-alpha shown as SEQ ID No. 2 on the inhibition of IL-13 from beagle leucocytes (stimulated with PHA).

FIG. 8 shows a comparison of the canine interferon-alpha shown as SEQ ID No. 1 with the synthetic interferon-alpha shown as SEQ ID No. 2 on the inhibition o production of IL-8 from beagle leucocytes.

FIG. 9 shows a comparison of the canine interferon-alpha shown as SEQ ID No. 1 with the synthetic interferon-alpha shown as SEQ ID No. 2 on inhibition of the production of IL-31 from beagle leucocytes with 0 stimulation, 4 μg/ml Con A plus 5 μl g/ml PWR and with 10 μg/ml PHA-P.

FIG. 10 shows a sequence alignment of SEQ ID NO: 1 and SEQ ID NO: 2 (A) and a sequence alignment of SEQ ID NO: 1 and SEQ ID NO: 3.

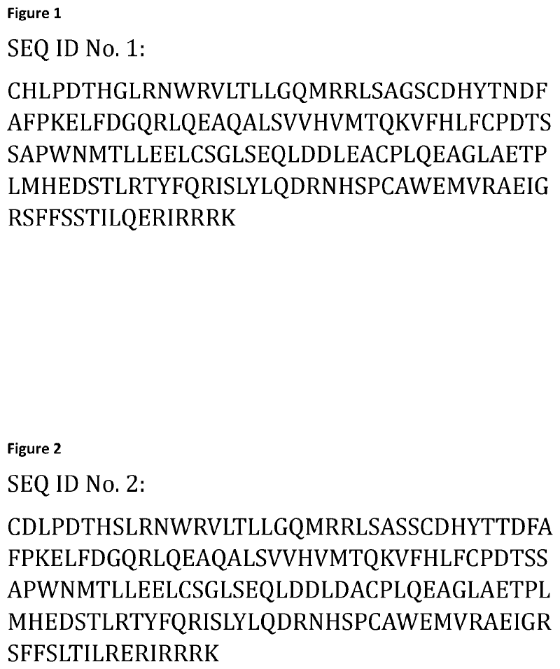

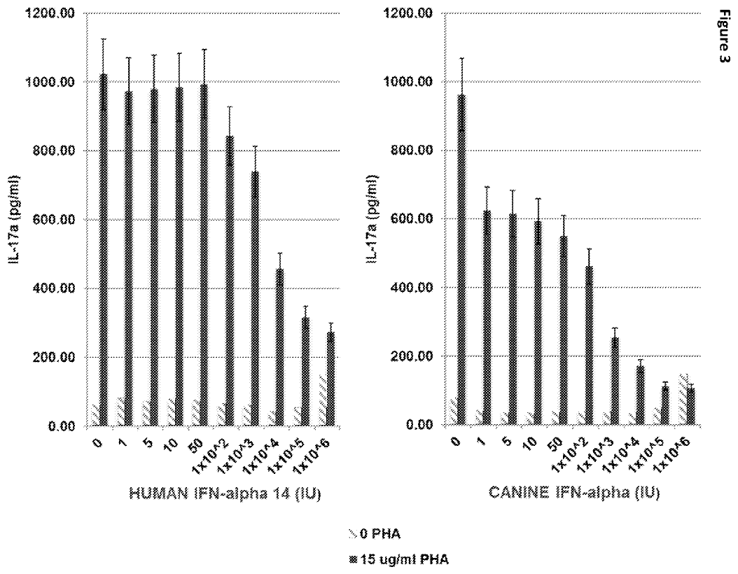

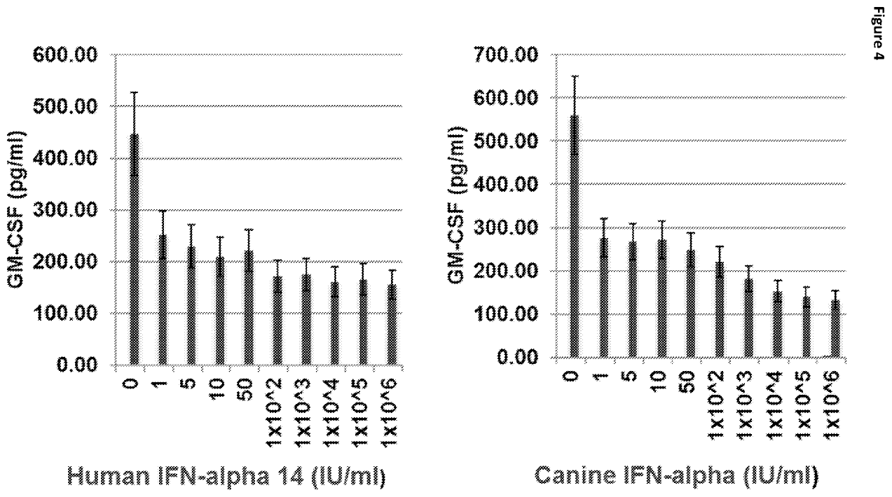

### EXPERIMENTAL DATA

Experiment 1: Comparison of the Canine Interferon-Alpha Shown as SEQ ID No. 1 with Human Interferon-Alpha 14 on IL-17A and GM-CSF in Beagle Whole Blood

Determination of Viability:

Fresh canine (Beagle) whole blood (Source: Tissue Solutions Ltd.) was diluted 1:100 with fresh RPMI 1640 culture medium containing 1% Penicillin/Streptomycin and 1% L-Glutamine—10 μl was added to 10 μl of 0.4% Trypan Blue solution and mixed. A further 10 μl was placed onto a Luna™ cell counting slide. The slide was then placed into the Luna-II™ Automated Cell Counter (Logos Biosystems) to determine the percentage viability of the cells by Trypan Blue exclusion.

Whole Blood Assay Protocol

Working within a Class II hood, whole canine blood containing 180 U/ml Sodium Heparin, diluted 1/10 with RPMI 1640 culture medium containing 1% Penicillin/Streptomycin and 1% L-Glutamine, was incubated with either no stimulus, 10 μg/ml phytohemagglutinin-P (PHA) or 4 μg/ml Concanavalin A from Canavalia ensiformis (ConA) and 5 μg/ml Lectin from Phytolacca americana (PWM), in the presence of a range of concentrations of either human rIFN-alpha 14 or canine rIFN-alpha (SEQ ID NO: 1) for 72 hours at 37° C. in an atmosphere of 5% CO₂ in air in a humidified incubator. IFN concentrations were 0, 1, 5 10, 50, 100, 1,000, 10,000, 100,000 and 1,000,000 IU/ml. Each culture concentration was 1 ml in a 24 well plate.

After 72 hours incubation, the samples are harvested by centrifugation at 10,000 g for 5 minutes to remove cells, the supernatants are carefully removed without disturbing the pellet of cells, and stored at −20° C. for future analysis. The supernatants are then assayed in relation to GM-CSF and/or IL-17a using the following ELISA methodologies as available in the art, for example, ELISA assays as available from R&D Systems, MN, USA

Typical ELISA Procedure Employed.

At least 16 hours prior to the start of the assay, 1 flat-bottomed 96 well microplate was coated with 100 μl the corresponding dilute capture antibody in phosphate buffer saline, without carrier protein. The plate was then sealed and incubated overnight at room temperature.

ELISA reagents and supernatants were allowed to warm to room temperature, and the pre-coated ELISA plate is washed three times with 400 μl wash buffer (1× phosphate buffer saline+0.05% Tween 20), and 300 μl reagent diluent was added to each well to prevent non-specific antibody binding, and incubated at room temperature for 1 hour.

The plate was again washed 3 times and 100 μl of diluted standards and samples were added to the appropriate wells. The plate was then sealed and incubated at room temperature for 2 hours with shaking (300-500 RPM).

After a further 3 wash cycles, 100 μl of Detection Antibody is added to each well, followed by a further 2-hour incubation at room temperature, with shaking.

The plate is washed a further 3 times, and 100 μl diluted Streptavidin-HRP solution was added to each well, followed by a 20-minute incubation at room temperature in darkness.

The plate was then washed a further 3 times, before adding 100 μl Substrate Solution for 20 minutes in darkness.

After 20 minutes, or when the standard wells reach the desired colour, 50 μl of stop solution 1M Sulphuric Acid and the plate was read via spectrophotometry, typically at wavelengths 450 and 570 nm.

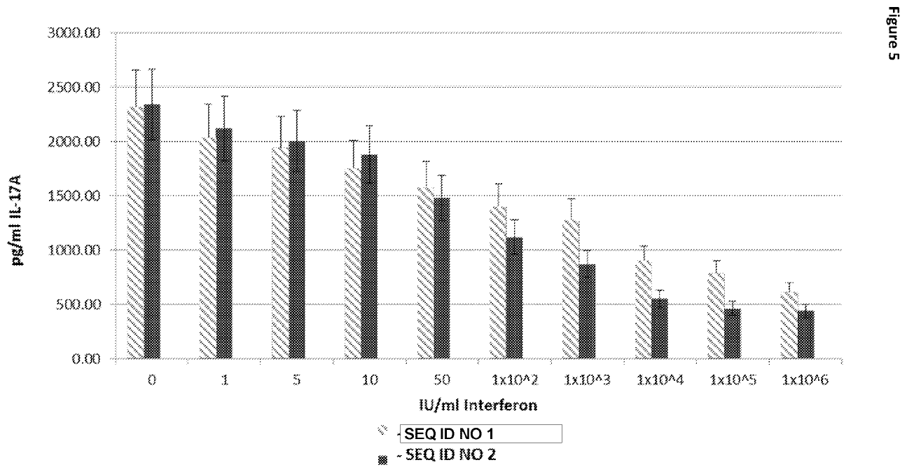

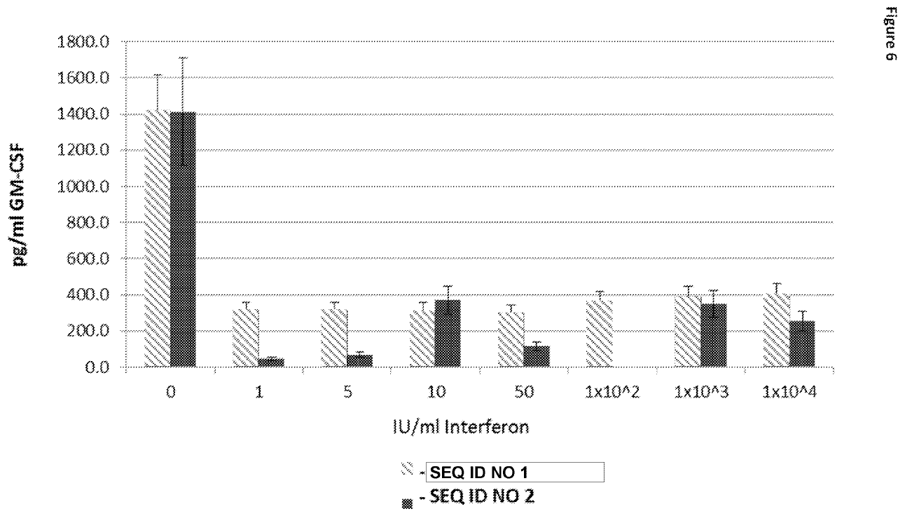

As shown in FIGS. 3 and 4, the particular canine interferon-alpha shown as SEQ ID No. 1 was superior to human alpha-14 in inhibiting cytokine IL-17A a GM-CSF response in beagle leucocytes.

Experiment 2: Comparison of the Canine Interferon-Alpha Shown as SEQ ID NO: 1 with the Synthetic Canine Alpha as Shown in SEQ ID NO: 2

Determination of Viability:

Fresh canine (Beagle) whole blood (Source: Tissue Solutions Ltd.) was diluted 1:100 with fresh RPMI 1640 culture medium containing 1% Penicillin/Streptomycin and 1% L-Glutamine—10 μl was added to 10 μl of 0.4% Trypan Blue solution and mixed. A further 10 dl was placed onto a Luna™ cell counting slide. The slide was then placed into the Luna-II™ Automated Cell Counter (Logos Biosystems) to determine the percentage viability of the cells by Trypan Blue exclusion.

Whole Blood Assay Protocol:

Working within a Class II hood, whole canine blood containing 180 U/ml Sodium Heparin, diluted 1/10 with RPMI 1640 culture medium containing 1% Penicillin/Streptomycin and 1% L-Glutamine, was incubated with either 15 μg/ml phytohemagglutinin-L (PHA), 100 μg/ml PHA or 10 μg/ml lipopolysaccharide (LPS) from Salmonella enterica serotype Abortus equi, in the presence of a range of concentrations of either canine rIFN (SEQ ID NO: 1) or canine rIFN-alpha (SEQ ID NO: 2) for 48 hours at 37° C. in an atmosphere of 5% CO₂ in air in a humidified incubator. IFN concentrations were 0, 1, 5 10, 50, 100, 1,000, 10,000, 100,000 and 1,000,000 IU/ml. Each culture concentration was 1 ml in a 24 well plate.

After 48 hours incubation, the samples are harvested by centrifugation at 10,000 g for 5 minutes to remove cells, the supernatants are carefully removed without disturbing the pellet of cells, and stored at −20° C. for future analysis. The supernatants are then assayed using the following ELISAs (following the manufacturer's instructions):
GM-CSF—R&D Systems, MN, USA
IL-13—Cloud-Clone Corp, TX, USA
   DLdevelop, Jiangsu, China
IL-22—BlueGene, Shanghai, China
IL-31—BlueGene, Shanghai, China
IL-17a—R&D Systems, MN, USA

Typical ELISA procedure employed.

ELISA reagents and supernatants were allowed to heat to room temperature, and the pre-coated ELISA plate is washed four times with wash buffer (1× phosphate buffer saline+0.05% Tween 20), and 200 μl assay diluent was added to each well to prevent non-specific antibody binding, and incubated at room temperature for 1 hour, with shaking (300-500 rpm, depending on protocol).

The plate was washed 4 times, and 100 μl of diluted standards and samples were added to the appropriate wells. The plate was then sealed and incubated at room temperature for 2 hours with shaking.

After a further 4 wash cycles, 100 μl of Detection Antibody is added to each well, followed by a further 1-hour incubation at room temperature, with shaking.

The plate is washed a further 4 times, and diluted Avidin-HRP solution is added to each well, followed by a 30-minute incubation at room temperature, with shaking.

The plate was then washed a total of 5 times, allowing for 30 seconds to 1 minute of soaking between washes, before adding 100 μl Substrate Solution for 15 minutes in darkness.

After 15 minutes, or when the standard wells reach the desired colour, 100 μl of stop solution 1M Sulphuric Acid and the plate was read via spectrophotometry, typically at wavelengths 450 and 570 nm.

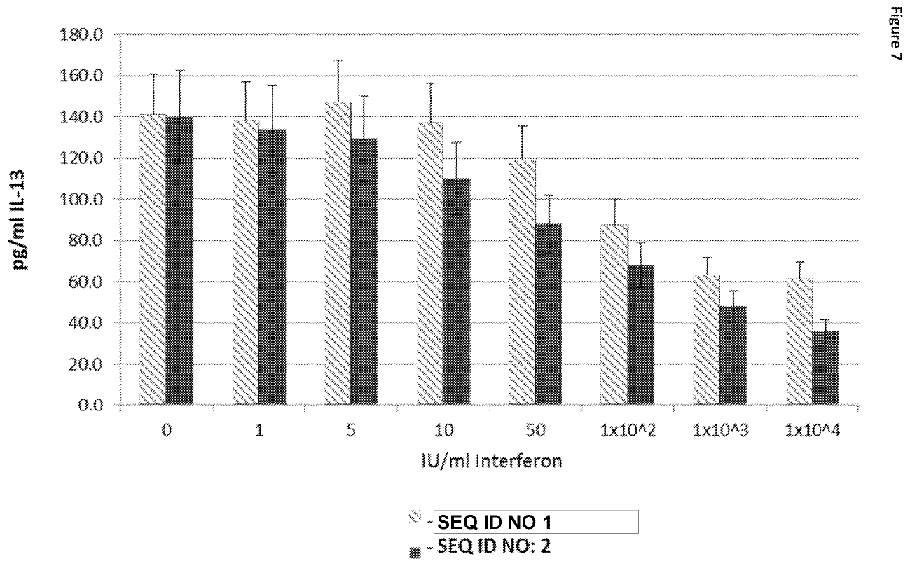

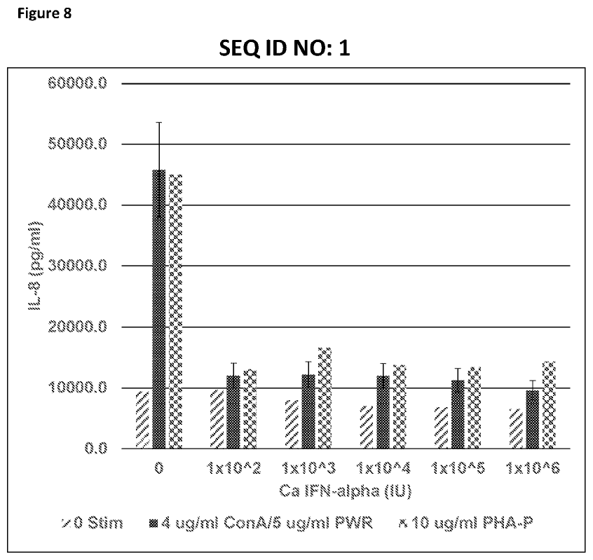

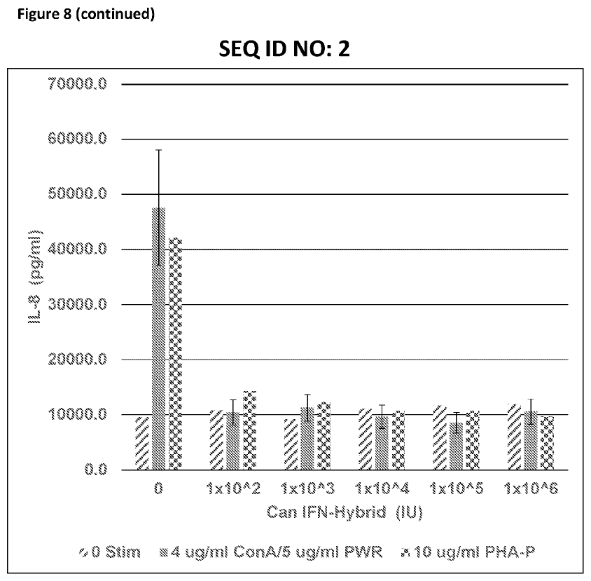

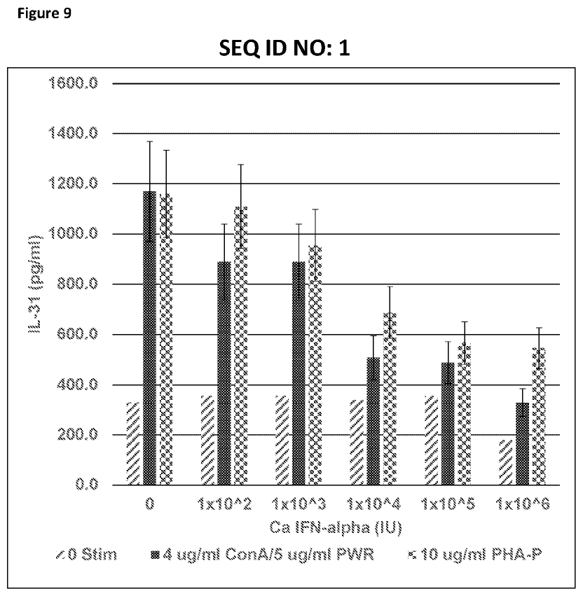

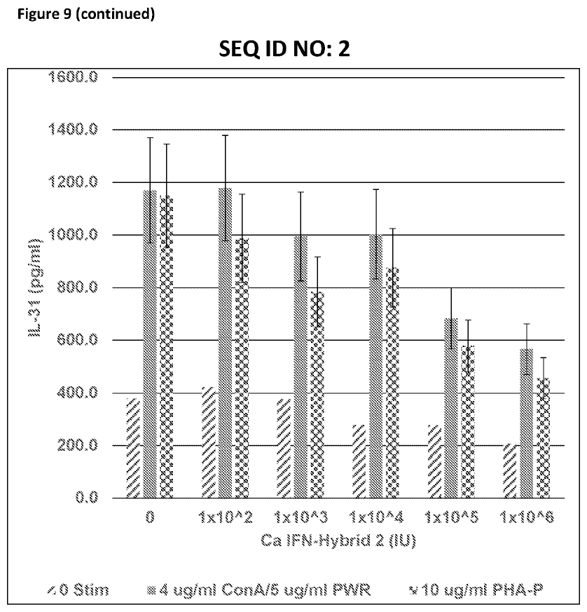

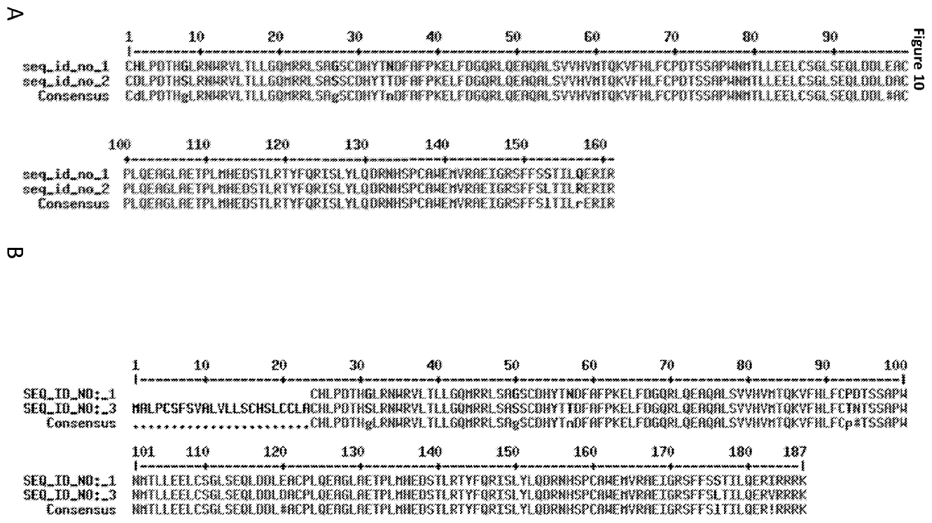

The results shown in FIGS. 5 to 9 demonstrate that the synthetic canine interferon-alpha shown as SEQ ID No. 2 is a functionally active variant of SEQ ID No. 1. Both SEQ ID No. 1 and SEQ ID No.2 displayed similar significant inhibitory effects on all the cytokines tested. These cytokines IL-17A, GM-CSF, IL-13, IL-8 and IL-31 have all been indicated as important markers in canine atopic dermatitis.

Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention, which are obvious to those skilled in the art, are intended to be covered by the present invention.

1. A canine interferon-alpha for use in the treatment or prophylaxis of canine pruritic disease, wherein the canine interferon-alpha comprises or consists of an amino acid sequence SEQ ID NO: 1 or a functionally active fragment or variant thereof wherein the variant comprises or consists of an amino acid sequence having at least 90% identity to SEQ ID NO: 1.

2. The canine interferon-alpha for use according to claim 1, wherein the canine pruritic disease is canine atopic dermatitis.

3. The canine interferon-alpha for use according to claim 1, wherein the variant has substitutions at one or more positions selected from the group consisting of: H2, G8, G27, N34, E97, S153, and Q157, wherein the position numbering is defined as that position which when aligned based on primary structure corresponds to the same position as that of SEQ ID NO: 1.

4. The canine interferon-alpha for use according to claim 1, wherein the variant comprises or consists of an amino acid sequence SEQ ID NO: 2, SEQ ID NO: 3 or a functionally active fragment of SEQ ID NO: 2 or SEQ ID NO: 3.

5. The canine interferon-alpha for use according to claim 1, wherein the variant comprises or consists of an amino acid sequence SEQ ID NO: 2, or a functionally active fragment of SEQ ID NO: 2.

6. The canine interferon-alpha for use according to claim 1, wherein the canine interferon-alpha is expressed in E. coli.

7. The canine interferon-alpha for use according to claim 1, wherein the canine interferon-alpha is formulated for sublingual, oral or subcutaneous administration.

8. A pharmaceutical composition for use in the treatment of canine pruritic disease, wherein the canine pruritic disease is canine atopic dermatitis, wherein the composition comprises a canine interferon-alpha as defined in claim 1 and a pharmaceutically acceptable carrier, diluent or excipient.

9. A method for the treatment and/or prophylaxis of canine pruritic disease, wherein the canine pruritic disease is canine atopic dermatitis, said method comprising the step of:
(i) administering to a canine in need thereof a therapeutically effective amount of a canine interferon-alpha comprising or consisting of an amino acid sequence SEQ ID NO: 1 or a functionally active fragment or variant thereof.

10. The method of claim 9, wherein the variant comprises or consists of an amino acid sequence having at least 90% identity to SEQ ID NO: 1 or a functionally active fragment of SEQ ID NO: 1.

11. The method of claim 9, wherein the variant has substitutions at one or more positions selected from the group consisting of: H2, G8, G27, N34, E97, S153, and Q157, wherein the position numbering is defined as that position which when aligned based on primary structure corresponds to the same position as that of SEQ ID NO: 1.

12. The method of claim 9, wherein the variant comprises or consists of an amino acid sequence SEQ ID NO: 2, SEQ ID NO: 3 or a functionally active fragment of SEQ ID NO: 2 or SEQ ID NO: 3.

13. The method of claim 9, wherein the variant comprises or consists of an amino acid sequence SEQ ID NO: 2, or a functionally active fragment of SEQ ID NO:2.

14. The method of claim 9, wherein the canine interferon-alpha was expressed in E. coli.

15. The method of claim 9, wherein the canine interferon-alpha is administered orally or by subcutaneous administration.

16. A method for the production of a canine interferon-alpha comprising the steps of:
(i) providing an Escherichia coli cell;
(ii) transforming the Escherichia coli cell with a heterologous nucleotide sequence encoding a canine interferon-alpha under the control of a promoter sequence wherein the canine interferon-alpha has at least 90% identity to SEQ ID NO:1 or a functionally active fragment of SEQ ID NO: 1; and
(iii) expressing the canine interferon-alpha in the cell.

17. A canine interferon-alpha for use in the treatment or prophylaxis of canine pruritic disease, optionally wherein the canine pruritic disease is canine atopic dermatitis, wherein the canine interferon-alpha is produced by the method of claim 16.

18. A method of treatment and/or prophylaxis of canine pruritic disease, optionally wherein the canine pruritic disease is canine atopic dermatitis, said method comprising the step of:
(i) administering to a canine in need thereof a therapeutically effective amount of a canine interferon-alpha produced by the method of claim 16.
